(19) United States
(12) Patent Application Publication
(10) Pub. No.: US 2006/0247544 A1
(43) Pub. Date: Nov. 2, 2006

(54) Characterization of cardiac motion with spatial relationship

(76) Inventors: Maleeha Qazi, King of Prussia, PA (US); Mustafa Kamasak, Kayseri (TR); Murat Dundar, Malvern, PA (US); Glenn Fung, Madison, WI (US); Sriram Krishnan, Exton, PA (US); R. Bharat Rao, Berwyn, PA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/332,975

(22) Filed: Jan. 17, 2006

Related U.S. Application Data
(60) Provisional application No. 60/649,794, filed on Feb. 3, 2005.

(51) Int. Cl. A61B 5/02

(52) U.S. Cl. 600/508

(57) ABSTRACT

Cardiac motion is automatically characterized based on spatial relationship to health. A classifier is trained for the characterization of cardiac motion. Regional wall motion abnormality assessment may be improved by combining information from neighboring segments. The structure or relationship between different segments and associated probabilities of different spatial locations being abnormal given another segment being abnormal are used for classification.

### RELATED APPLICATIONS

The present patent document claims the benefit of the filing date under 35 U.S.C. §119(e) of Provisional U.S. Patent Application Ser. No. 60/649,794, filed Feb. 3, 2005, which is hereby incorporated by reference.

### BACKGROUND

This present invention relates to characterizing cardiac motion. Cardiac wall motion abnormalities are often used as a surrogate marker for cardiac ischemic disease. A number of different imaging modalities can be used to study or diagnose wall motion abnormalities, including ultrasound, MRI, CT, nuclear medicine, and angiography. Typically, wall motion abnormalities are assessed by a trained physician. A sequence of images is analyzed by the physician. Wall motion abnormalities may be assessed quantitatively, such as disclosed in U.S. Pat. No.______ (U.S. Published Application No. 20050059876), the disclosure of which is incorporated herein by reference.

A number of features are used to characterize the cardiac motion in order to detect cardiac motion abnormalities. For example, ejection-fraction ratio, radial displacement, velocity, thickness and thickening are used. Typically, wall motion analysis is done by dividing the heart into a number of segments. For example in ultrasound, the heart is often divided into 16 or 17 segments. A “bulls-eye” representation of the segmented heart is shown in FIG. 1. Each segment is then scored, for example on a scale from 1-5, denoting the level or type of abnormalities.

However, such segmentation is often artificial. That is, if one segment is abnormal, then it is likely that neighboring segments are abnormal as well. This is particularly true for neighboring segments that are fed from the same coronary arteries. If a coronary artery is blocked, causing ischemia, then all of the segments fed by that artery may move abnormally. This information is often used by the cardiologist in assessing wall motion abnormalities.

### BRIEF SUMMARY

By way of introduction, the preferred embodiments described below include methods, computer readable media and systems for characterizing cardiac motion based on spatial relationship and for training a classifier for characterizing cardiac motion. Regional wall motion abnormality assessment may be improved by considering health information from neighboring segments. The structure or relationship between different segments and associated probabilities of different spatial locations being abnormal given another segment being abnormal are used for classification.

In a first aspect, a method is provided for characterizing cardiac motion based on spatial relationship. A sequence of images representing a heart wall as a function of time is obtained. The heart wall is segmented into a plurality of segments. A processor implementing a classifier assesses the heart wall as a function of the segments. The classifier incorporates health dependency between segments.

In a second aspect, a computer readable storage media has stored therein data representing instructions executable by a programmed processor for characterizing cardiac motion based on spatial relationship. The storage media includes instructions for classifying cardiac motion as a function of a first likelihood of a first location being abnormal if a second location is abnormal and outputting the classification.

In a third aspect, a system is provided for characterizing cardiac motion based on spatial relationship. A memory is operable to store a sequence of images representing a heart wall as a function of time and operable to store domain knowledge of a relationship of heart wall health of different segments with each other. A processor is operable to characterize cardiac motion of the heart wall with a classifier from the sequence of images. The classifier is responsive to the domain knowledge.

In a fourth aspect, a method is provided for training a classifier of cardiac wall motion based on spatial relationships. Dependencies between heart wall segments are learned based on known scores for test cases. The dependencies are incorporated into a Bayesian classifier as a prior distribution with probabilities. The probabilities are prior domain knowledge. The classifier is generated by the incorporation.

In a fifth aspect, a method is provided for training a classifier of cardiac wall motion based on spatial relationships. A processor learns a segment health relationship. The processor then learns based on the segment health relationship and segment feature. The classifier is generated from the learning acts.

In a sixth aspect, a method is provided for training a first classifier of cardiac wall motion based on spatial relationships. A probabilistic model of health relationships between heart wall segments is determined. A second classifier of the heart wall segments is trained with the training being independent of the probabilistic model. The first classifier is generated with the probabilistic model and outputs of the second classifier.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a bulls-eye graphical representation of segmentation of a heart wall;

FIG. 2 is a block diagram of one embodiment of a system for characterizing cardiac motion base, in part, on spatial health relationship;

FIG. 3 is a flow chart diagram of one embodiment of a method for characterizing cardiac motion based, in part, on spatial health relationship;

FIG. 4 is a graphical representation of one embodiment of a dependency structure of segments; and

FIG. 5 is a graphical representation of one embodiment of a dependency structure of segments and associated segment features for classification.

### DETAILED DESCRIPTION OF THE DRAWINGS AND PRESENTLY PREFERRED EMBODIMENTS

The relationship of health of different segments to other segments is incorporated in automatic assessing of wall motion abnormalities. Domain knowledge of the dependency structure between the segments and the corresponding probabilities are incorporated. This relationship of health of one segment to other segments is learnt from training data and/or provided as a prior known relationship. The causal health relationships may mirror the actual physical relationships within the heart.

The system, methods and instructions herein may instead or additionally be used for other interrelated motion characterization, such as analysis of diaphragm motion or a gait while jogging. In yet other embodiments, non-medical analysis is performed using the methods, systems, or instructions disclosed herein, such as analysis of turbine blade vibrations or structural reaction to environmental conditions (e.g., bridge variation due to wind). A medical imaging cardiac motion example is used herein.

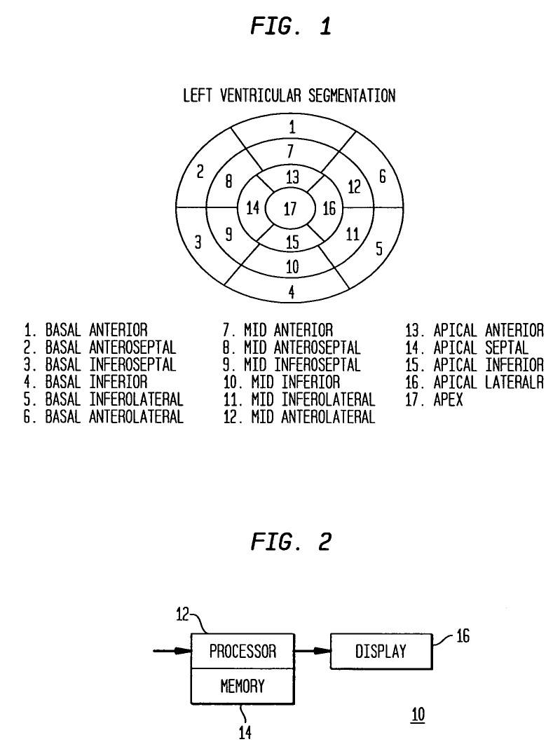

FIG. 1 shows a system 10 for characterizing cardiac motion based on spatial relationship. The system 10 includes a processor 12, a memory 14 and a display 16. Additional, different or fewer components may be provided. In one embodiment, the system 10 is a medical diagnostic imaging system, such as an ultrasound imaging system. As or after images representing a patient's heart are acquired, the system 10 automatically characterizes the cardiac motion of the heart. In other embodiments, the system 10 is a computer, workstation or server. For example, a local or remote PACs workstation receives images and characterizes cardiac motion.

The memory 14 is a computer readable storage media. Computer readable storage media include various types of volatile or non-volatile storage media, including but not limited to random access memory, read-only memory, programmable read-only memory, electrically programmable read-only memory, electrically erasable read-only memory, flash memory, magnetic tape or disk, optical media, database, and the like. The memory 14 may include one device or a network of devices with a common or different addressing scheme. In one embodiment, a single memory 14 stores image data, domain knowledge, a classifier and instructions for operating the processor 12, but separate storage may be provided for one or more types of data.

The memory 14 stores medical image data for or during processing by the processor 12. For example, ultrasound data representing a sequence of B-mode images of a myocardium at different times is stored. The images are stored in a CINE loop, DICOM or other format. The sequence of images represent a heart wall as a function of time. A single sequence or a plurality of sequences are stored. The sequence is for classification. Alternatively or additionally, the sequence is one of a plurality of sequences and associated diagnosis (i.e., truth values) of a training data set for training a classifier.

The memory 14 stores domain knowledge of a relationship of heart wall health of different segments with each other. The relationship is a network of dependencies and/or associated probabilities. For example, segments 3 and 9, the basal and mid inferoseptal walls, are linked physiologically in patients. The reason is that both walls are fed by the same coronary artery, the left anterior descending artery (LAD). As a result, any disease in the LAD may affect both of these walls together. The relationship is probabilistic for a couple of reasons. First, if the disease is not in the main LAD, but in a particular branch, it may affect only one of the two walls. Also, some patients may have abnormal coronary anatomy where one or both of these walls are fed by a different coronary artery. However, in general many such relationships occur. Any threshold amount of relationship may be used, such as associated with any, 1, 5, 10, 20 or other percentage of likelihood.

The domain knowledge is part of a classifier, model, prior knowledge, or combinations thereof. For example, the relationship is a learned causal relationship between the different segments. The domain knowledge may be part of a trained classifier. The training includes relationship structure and/or probabilities considerations. The domain knowledge may be a model, such as a graphical probabilistic model, used to train a classifier or used by a classifier for diagnosis. As another example, the domain knowledge is a prior distribution used by the classifier. The prior distribution is obtained from research, doctors or other sources. Related or linked segments and/or the corresponding likelihood are known, so the prior distribution is coded as a look-up table, algorithm or other data. The prior distribution is then used by the classifier or for training the classifier.

The processor 12 is one or more general processors, digital signal processors, application specific integrated circuits, field programmable gate arrays, servers, networks, digital circuits, analog circuits, combinations thereof, or other now known or later developed device for classifying medical image data. The processor 12 implements a software program, such as code generated manually (i.e., programmed) or a trained or training classification system. For example, the processor 12 is a classifier implementing a graphical model (e.g., Bayesian network, factor graphs, chain graph, or hidden or random Markov models), a boosting base model, a decision tree, a neural network, combinations thereof or other now known or later developed algorithm or classifier. The classifier is configured or trained for distinguishing between the desired groups of states or to identify options and associated probabilities.

In one embodiment, the processor 12 is operable to characterize cardiac motion of the heart wall from the sequence of images with a classifier. Features are extracted from the medical images automatically or input manually. The classifier assesses the heart wall function based on the features. The classifier indicates abnormal, normal or scores for segments or an entire heart. One method characterizes the motion of each segment of the heart on a scale of 1-5, as per guidelines from the American Society of Echocardiography. The classifiers disclosed in U.S. Patent Publication Nos. 20050059876 or 20040208341, the disclosures of which are incorporated herein by reference, may be used.

In one embodiment, the processor 12 implements a model or trained classification system (i.e., the processor is a classifier) programmed with desired thresholds, filters or other indicators of class. For example, the processor 12 or another processor tracks one or more points and calculates spatial parameter values for each point in a first level of a hierarchal model. The processor 12 then characterizes the cardiac motion as a classifier with the spatial parameter values being used for inputs in a second level of the hierarchal model. As another example, the processor 12 is implemented using machine learning techniques, such as training a neural network using sets of training data obtained from a database of patient cases with known diagnosis. The processor 12 learns to analyze patient data and output a diagnosis. The learning may be an ongoing process or be used to program a filter or other structure implemented by the processor 12 for later existing cases. Any now known or later developed classification schemes may be used, such as cluster analysis, data association, density modeling, probability based model, a graphical model, a boosting base model, a decision tree, a neural network or combinations thereof.

The assessment by the classifier is responsive to the domain knowledge indicating the health relationship of different spatial locations or segments. The output of the classifier is based, in part, on which segments are associated and/or on the likelihood of the associated segments having a common health. For example, the processor 12 classifies each segment independently, and then classifies the segments based on the independent classification and the health relationship domain knowledge. As another example, the classifier implements a model including the relationship information and input features in a single level. The domain knowledge is learned, such as parameters from machine training, or programmed based on studies or research. The domain knowledge may be disease, institution, or user specific, such as including procedures or guidelines implemented by a hospital. The domain knowledge may be parameters or software defining a learned model.

The memory 14 stores data representing instructions executable by a programmed processor, such as the processor 12, for automated analysis of heart function based on the domain knowledge. The automatic or semiautomatic operations discussed herein are implemented, at least in part, by the instructions. In one embodiment, the instructions are stored on a removable media drive for reading by a medical diagnostic imaging system or a workstation networked with imaging systems. An imaging system or workstation uploads the instructions. In another embodiment, the instructions are stored in a remote location for transfer through a computer network or over telephone communications to the imaging system or workstation. In yet other embodiments, the instructions are stored within the imaging system on a hard drive, random access memory, cache memory, buffer, removable media or other device.

The functions, acts or tasks illustrated in the figures or described herein are performed by the programmed processor 12 executing the instructions stored in the memory 14 or a different memory. The functions, acts or tasks are independent of the particular type of instructions set, storage media, processor or processing strategy and may be performed by software, hardware, integrated circuits, film-ware, micro-code and the like, operating alone or in combination. Likewise, processing strategies may include multiprocessing, multitasking, parallel processing and the like.

In one embodiment, the memory 14 is a computer readable storage media having stored therein data representing instructions executable by the processor 12 for characterizing cardiac motion with spatial health relationship information. The instructions cause the processor 12 to characterize cardiac motion as a function of spatial health relationships. The instructions are for any or some of the functions or acts described herein. For example, in response to the instructions, the processor 12 segments data representing a heart wall, such as into 2 or more (e.g., 15-17) segments. Thresholding, edge enhancement, manual input, motion tracking and/or other techniques identify the heart wall throughout the sequence. The heart wall is then divided into equally spaced segments, user identified segments or segments corresponding to particular structure. The segmentation is automatic, responsive to user input or semiautomatic.

The instructions include operations for receiving input of a plurality of features for each of the segments into the classifier. The inputs are provided automatically, such as determining one or more features with an algorithm, or manually, such as a user inputting the features. Any one or more features and/or extraction techniques may be used, such as the features and techniques disclosed in U.S. Patent Publication Nos. 20050059876 or 20040208341. In one embodiment, the local ejection fraction, displacement, radial displacement, timing, velocity, timing amplitude, fractional shortening, Eigen motion, strain, radial strain, thickening, combinations thereof or other features are used. The same or different features may be used for different segments.

In order to calculate the above or other feature values as a function of time, the image data associated with particular time periods is identified. For example, ECG information is used to identify data associated with one or more portions of or whole heart cycles. As another example, Doppler acceleration, velocity or power data is analyzed to identifying the heart cycle timing and associated data.

The instructions include operations for classifying cardiac motion as a function of a likelihood of one location being abnormal if another location is abnormal. The likelihood may be associated with two or more options, such as normal or abnormal, or normal or particular abnormality. The classification uses a learned causal relationship between the different spatial locations, such as a network of health dependencies between pluralities of segments. The network structure and/or likelihoods within the network are used. Different pairs or groupings of segments may have sufficient relationships with respect to health to be included in the network. The level of relationship provides a likelihood of similarity or dissimilarity.

The classifying is based on the features and the relationship structure and/or likelihood. Different segments are scored as normal, abnormal or having a particular abnormality. In some embodiments, the likelihood used in the scoring is a prior probability programmed for use in a learned structure or a prior known structure. In other embodiments, the likelihood is learned using a prior known structure. In other embodiments, the likelihood is a function of a model, such as a graphical probabilistic model. The features and the graphical probabilistic model are used to classify the heart wall. In other embodiments, the likelihood is also a function of a model. Each segment is independently classified. The outputs of the independent classification and the model are used to classify the heart wall.

The instructions cause the processor 12 to output classification results. For example, the results are displayed a numbers, text, a graph or an image on the display 16. The results may be stored in the memory 14.

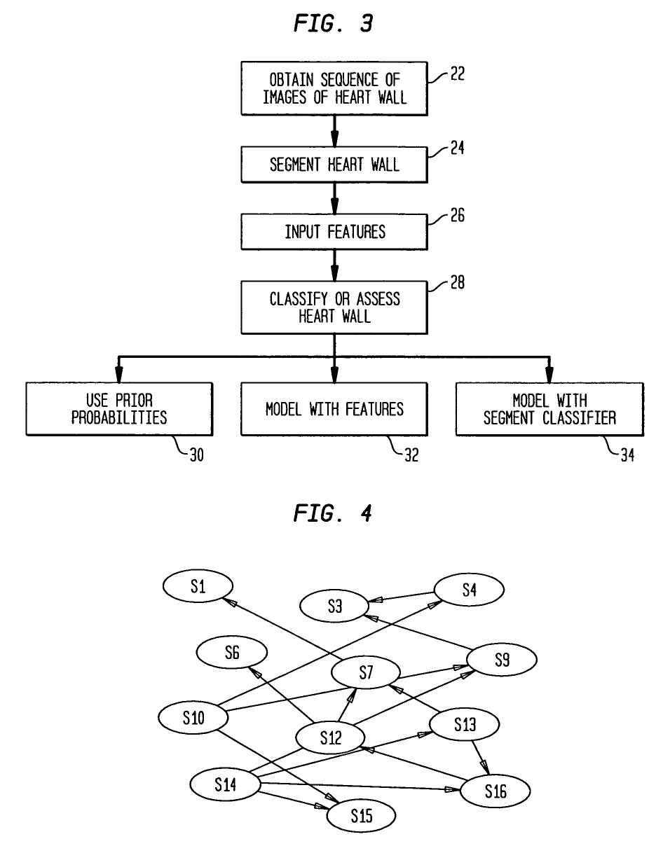

FIG. 3 shows one embodiment of a method for characterizing cardiac motion based on spatial health relationship. The method is implemented by the system 10 of FIG. 2 or a different system. Each act is performed automatically with a processor, but one or more acts may be performed manually or with manual input. The acts are performed in the order shown or a different order. Additional, different or fewer acts may be provided.

In act 22, a sequence of images representing a heart wall as a function of time is obtained. The sequence is obtained in real-time, such as by scanning a patient. Alternatively, the sequence is obtained from a previous examination, such as from an archival system. The sequence corresponds to any length of time or event, such as one or more heart cycles. The images of the sequence may be decimated or increased by interpolation. Different or the same processing of the images may be provided for different images in a sequence or for different sequences. In one embodiment, the sequence of images includes data representing a heart wall without textual or other overlay data. Alternatively, textual or overlay data is removed by processing or the images used for classification include textual and/or overlay information.

In act 24, the heart wall is segmented into a plurality of segments. Any number of segments may be used, such as the 17 segments shown in FIG. 1. Different segments may be used for different views or partial views of the heart wall. Each segment corresponds to a same or different number of spatial location samples, such as each segment having a same area or volume. The segments are equally or unequally spaced along the heart wall. Automated, manual or semi automated (automatic with manual guidance) segmentation may be used. For example, the user indicates beginning and ending points of a heart wall in one image. The heart wall is determined by thresholding or region growing based on the identified beginning and ending locations. The segments are automatically defined as equally spaced along the heart wall within the image. For subsequent images, the segments are tracked from one image to the next using correlation of features or speckle.

In act 26, a plurality of feature values are input for each of the segments. The same or different features may be used for each segment. The features are extracted automatically, manually or a combination of automatically and manually. The features are extracted from the sequence of images. Features may be obtained from other sources, such as data mining or manually inputting heart rate, age, previous diagnosis, clinical or test result information. The features are input into the classifier.

In act 28, the heart wall is assessed as a function of the segments. A processor implementing a classifier classifies the heart wall segments. In alternative embodiments, the heart wall is assessed globally instead of as a function of the segments.

The classifier incorporates health dependency between segments. A relationship of abnormal or normal operation (e.g., motion) of each of the segments with abnormal or normal operation of other segments may increase accuracy of heart wall assessment. Where one segment is abnormal or normal, the likelihood of abnormal or normal operation of one or more other segments may be greater or lesser. The health relationship identifies the related segments (structure) and the associated probabilities. Classification is a function of the health dependency. The health dependency is learned causal relationships between the segments. For example, the health dependency is learned from training data. Alternatively, a portion or all of the health dependency is provided as prior knowledge.

The health dependency for classification may be incorporated in any now known or later developed method, such as maximum likelihood classification. Domain knowledge of the health dependency is incorporated into the classifier. The casual relationships of the health dependency may be similar to the actual physical relationships in the heart, such as segments associated with a same artery being more likely normal or abnormal as a group. Acts 30, 32, and 34 show three alternative methods for incorporating the health dependency. Other method may be used.

In act 30, the classifier incorporates the health dependency as prior information. The structure and/or the probabilities are prior knowledge. In one embodiment, the classifier incorporates a learned dependency between classified segment scores based on prior probabilities. For example, the classifier is trained to classify cardiac wall motion based on spatial relationships by learning dependencies between heart wall segments based on known scores for test cases. Alternatively, the dependency structure is based on prior knowledge, such as the artery structure of the heart wall, and the probabilities are learned.

The probabilities corresponding to the dependency structure may be learned from the labels. Training data with prior provided scores, indications of normal/abnormal or other truth values (i.e., labels) are input. Any classification technique (e.g. neural networks, SVM, or Fisher's discriminant) learns the probabilities of the dependencies between the segments based on the training data. In this classification task, the labels considered are not only the labels of the segments to be classified but the labels of neighboring segments as well. The classifier finds the relations of the labels of each neighboring segment with the label of the segment to be classified. Broader, such as non-neighboring segments, or narrower, such as less than all neighboring segments, may be used. Alternatively, the probabilities are prior domain knowledge, such as provided by research or manual doctor input.

The health dependencies (e.g., the structure and probabilities associated with the structure) are incorporated into a classifier, such as a Bayesian classifier. The learned or prior spatial health relationships are incorporated into a Bayesian classifier as a prior distribution and a final classifier is designed from the same or different training data. The incorporation is used to generate the classifier. Other classifiers may be used, such as a classifier based on maximum likelihood.

In act 32, the classifier incorporates the health dependency with a model, such as a graphical probabilistic model. The graphical probabilistic model is of any now known or later developed model, such as a Bayesian Network, random Markov fields, chain graphs, undirected graphical model, optimal tree structure for Bayesian models (e.g., network models using the minimum spanning tree algorithm, mutual information between feature pairs for structure, and belief propagation for inference (likelihood)).

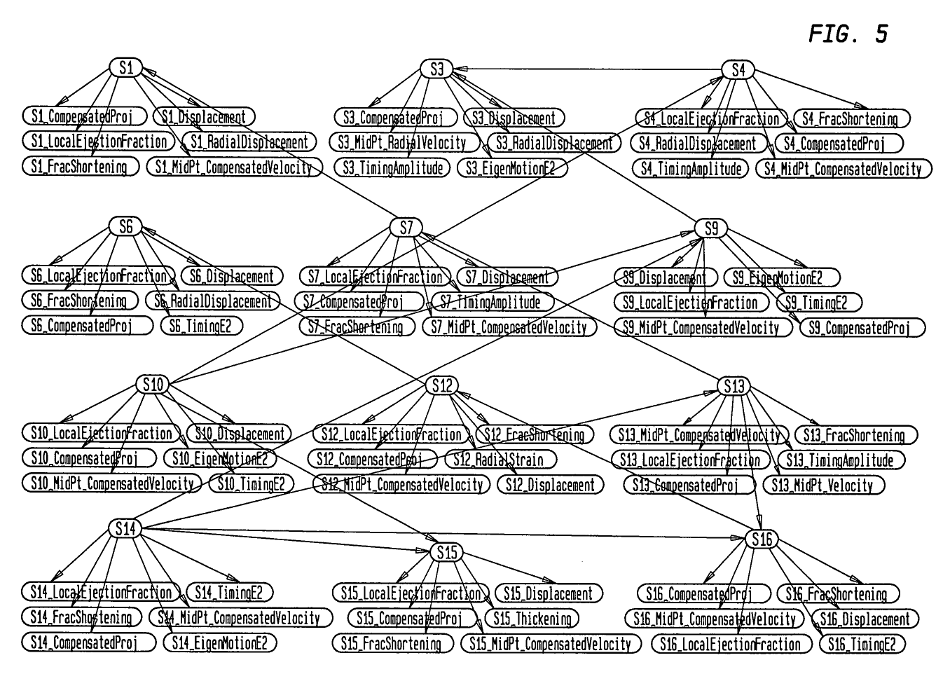

The processor implementing the classifier of cardiac wall motion learns based, in part, on spatial relationships. A processor learns segment health relationships (e.g., the relationship of health between segments). The health dependencies may be learned as part of a single process or a hierarchal process. For example, the structure is learned assuming possible connections from each segment to all other segments. The learning method looks for strong and weak relationships. The probabilities for the structure are then learned from the same training data or different training data as a function of the previously learnt structure. FIG. 4 shows one example graphical model of a learnt Bayesian network which involves the 12 segments from Apical 4-chamber and Apical 2-chamber views. A greater or lesser number of segments may be used, such as including 17 segments.

In one embodiment, the structure is learnt using Hugin Expert's Necessary Path Condition (NPC) algorithm or a Bayesian Network Toolbox. The algorithm relies on user interaction to resolve inconsistencies. The background knowledge used to determine the final solution comes from doctors and is that segments that are neighbors influence one another, especially if they are fed by the same coronary artery. Learnt relationships between neighboring segments that share the same artery feed receive higher preference.

The resulting graphical model with some additional features is used to provide segment level or heart level classification. A processor learns to classify based on the graphical model representing the segment health relationship and features. Various features may be used, such as segment or global features. In one example embodiment, the network of FIG. 4 was added to manually to test different combinations of local and global features to improve classification accuracy. FIG. 5 shows an example that includes the six local features per segment based on Kolmogorov Smirnov Statistics. Different, additional or fewer features, such as other segment features or global features, may be used. For example, velocity, regional ejection fraction, and fractional shortening are used. Fractional shortening is the % change between two points along the contour within a segment from end-diastole to end-systole. The classifier is generated by learning from the training set using the desired features and the graphical model.

In act 34 of FIG. 3, a model of the health dependencies is used. A model, such as a probabilistic model of health relationships between heart wall segments, is determined. The model is determined as discussed above for act 32.

Instead of training a single classifier based on the model and features, separate classifiers are trained. In a first level of classifier training, a classifier is trained for each of the segments. For each segment, the classifier is trained independently using any classification technique (e.g. neural networks, SVM, or Fisher's discriminant). A same classifier may be used for two, more, or all of the segments. The segment classifier or classifiers are independent of the spatial health relationship model. Each segment is assessed independently with a same or different features and/or classifiers.

Another classifier is then generated using the model and the outputs of the segment classifier or classifiers. The trained graphical model representing the dependency structure between the segments and the independently trained classifier outputs are used to predict labels for each segment. Each segment classifier's output is an “effect” of the final segment label. This allows for inference of the segment labels from the graphical model given the segment classifier outputs. The health dependency in incorporated as a function of a dependency model and the independent segment assessments.

In act 28, one or more classifiers developed in acts 30, 32 and/or 34 assess the heart wall motion for a test or input image or sequence of images. The assessment is based on features extracted from the images and the health dependency between segments. Each of the segments is scored or indicated as normal or abnormal. Alternatively or additionally, an overall score or health indication of the heart wall is output.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for characterizing cardiac motion based on spatial relationship, the method comprising:
obtaining a sequence of images representing a heart wall as a function of time;
segmenting the heart wall into a plurality of segments;
assessing, with a processor implementing a classifier, the heart wall as a function of the segments, the classifier incorporating health dependency between segments.

2. The method of claim 1 wherein assessing comprises assessing with the classifier, the classifier incorporating probabilities of heart wall segment performance as a function of the health dependency.

3. The method of claim 1 wherein assessing comprises assessing with the classifier, the classifier incorporating learned causal relationships between the segments.

4. The method of claim 3 wherein assessing comprises assessing with the classifier, the health dependency learned from training data.

5. The method of claim 1 further comprising:
inputting a plurality of features for each of the segments into the classifier;
wherein the assessing is based on the features and the health dependency between segments.

6. The method of claim 1 wherein assessing comprises assessing with the classifier incorporating the health dependency, the health dependency comprising a relationship of abnormal operation of each of the segments with abnormal operation of all of the other segments, likelihood of abnormal operation being greater with abnormal operation of some of the other segments based on the relationship.

7. The method of claim 1 wherein assessing comprises scoring each of the segments.

8. The method of claim 1 wherein assessing comprises assessing with the classifier, the classifier incorporating the health dependency as a learned probability based on prior dependency structure.

9. The method of claim 1 wherein assessing comprises assessing with the classifier, the classifier incorporating the health dependency with a graphical probabilistic model.

10. The method of claim 1 wherein assessing comprises assessing with the classifier, the classifier assessing each segment independently and incorporating the health dependency as a function of a dependency model and the independent segment assessments.

11. In a computer readable storage media having stored therein data representing instructions executable by a programmed processor for characterizing cardiac motion based on spatial relationship, the storage media comprising instructions for:
classifying cardiac motion as a function of a first likelihood of a first location being abnormal if a second location is abnormal; and
outputting the classification.

12. The instructions of claim 11 wherein classifying comprises classifying as a function of a network of health dependencies between a plurality of spatial locations including the first and second spatial locations.

13. The instructions of claim 12 wherein classifying comprises classifying as a function of a plurality of likelihoods including the first likelihood, the each of the plurality of likelihoods corresponding different combinations of spatial locations within the network.

14. The instructions of claim 11 further comprising:
segmenting data representing a heart wall, the first location corresponding to a first segment and the second location corresponding to a second segment different from the first segment.

15. The instructions of claim 11 wherein classifying comprises classifying with a learned causal relationship between the first and second locations.

16. The instructions of claim 11 wherein the first and second locations correspond to first and second segments of a heart wall;
further comprising:
inputting a plurality of features for each of the segments into the classifier;
wherein the classifying is based on the features and the likelihood.

17. The instructions of claim 11 wherein classifying comprises scoring the first location and the second location, the score indicating normal or abnormal.

18. The instructions of claim 11 wherein classifying comprises classifying with the likelihood being a prior structure for a learned probability.

19. The instructions of claim 11 wherein classifying comprises classifying with the likelihood being a function of a graphical probabilistic model.

20. The instructions of claim 11 wherein classifying comprises classifying each location independently and classifying as a function of the likelihood and the independent location classifications.

21. A system for characterizing cardiac motion based on spatial relationship, the system comprising:
a memory operable to store a sequence of images representing a heart wall as a function of time and operable to store domain knowledge of a relationship of heart wall health of different segments with each other;
a processor operable to characterize cardiac motion of the heart wall with a classifier from the sequence of images, the classifier responsive to the domain knowledge.

22. The system of claim 21 wherein the relationship comprises a network of dependencies and associated probabilities.

23. The system of claim 21 wherein the relationship comprises a learned causal relationship between the different segments.

24. The system of claim 21 wherein the domain knowledge is a prior distribution used by the classifier.

25. The system of claim 21 wherein the domain knowledge is a graphical probabilistic model used by the classifier.

26. The system of claim 21 wherein the classifier is operable to classify each segment independently and classify as a function of the relationship and the independent segment classifications.

27. A method for training a classifier of cardiac wall motion based on spatial relationships, the method comprising:
learning probabilities between heart wall segments based on known scores for test cases;
incorporating the dependencies into a Bayesian classifier as a prior distribution with a structure, the structure being prior domain knowledge; and
generating the classifier by the incorporation.

28. A method for training a classifier of cardiac wall motion based on spatial relationships, the method comprising:
learning with a processor a segment health relationship; then
learning with the processor based on the segment health relationship and segment features; and
generating the classifier from the learning acts.

29. The method of claim 28 wherein learning the segment health relationship comprises learning a structure and then learning probabilities for the structure as a function of the structure.

30. A method for training a first classifier of cardiac wall motion based on spatial relationships, the method comprising:
determining a probabilistic model of health relationships between heart wall segments;
training a second classifier of the heart wall segments, the training being independent of the probabilistic model; and
generating the first classifier with the probabilistic model and outputs of the second classifier.
